(19) United States
(12) Patent Application Publication
(10) Pub. No.: US 2002/0155192 A1
(43) Pub. Date: Oct. 24, 2002

(54) Yeast transport isoforms

(76) Inventors: John Walker, Cambridge (GB); Michael Runswick, Cambridge (GB); Ferdinando Palmieri, Bari (IT); Luigi Palmieri, Bari (IT); Gennaro Agrimi, Bari (IT)

Correspondence Address: HALE & DORR LLP, THE WILLARD OFFICE BUILDING, 1455 PENNSYLVANIA AVE, NW, WASHINGTON, DC, 20004, US

(21) Appl. No.: 10/042,194

(22) Filed: Jan. 11, 2002

Related U.S. Application Data
(63) Continuation-in-part of application No. PCT/GB01/03451, filed on Aug. 1, 2001.

(30) Foreign Application Priority Data
Aug. 1, 2000 (GB) 0018880.5
Feb. 13, 2001 (GB) 0103537.7

(51) Int. Cl. A21D 8/00; C12N 1/18; C12P 13/08

(52) U.S. Cl. 426/27; 435/115; 435/254.2

(57) ABSTRACT

A method is provided for modulating transport of an oxodicarboxylate across the mitochondrial membrane of a yeast which method comprises modulating in said yeast the activity of one or more Odc1p and/or Odc2p yeast mitochondrial transport polypeptides.

### FIELD OF THE INVENTION

The present invention relates to methods for modulating the transport of metabolic intermediates across the mitochondrial membrane in yeasts., particularly to modulate the rate of lysine biosynthesis in said yeasts.

### BACKGROUND OF THE INVENTION

L-Lysine is an essential amino acid and is used in large quantities as animal feed supplement. Numerous amino acids are generally produced biosynthetically by bacterial fermentation processes which have known and used in the art for many years. The bacterial strains for producing amino acids are distinguished by their capacity for secreting these amino acids into the culture medium at high concentrations within a short time.

Single cell protein, derived from yeasts, has also been used to supplement animal feed. Further, attempts have been made to improve the nutritive properties of human food products such as bakery products that require yeast in their manufacture by increasing the degree of lysine production by increasing the lysine production by said yeast.

Thus, as an alternative, or in addition to, the use of bacterially-produced lysine in animal feed supplements, it would be desirable to use yeasts that over-produce lysine as a constituent of such animal feed supplements. Also, it may be desirable to modify yeasts that are already used in human or animal food production, such as Saccharomyces cerevisiae, such that they over-produce lysine and thus increase the nutritive value of those foods by increasing the content of lysine, an essential amino acid.

### SUMMARY OF THE INVENTION

The nuclear genome of S. cerevisiae encodes 35 members of a family of membrane proteins. Known members transport substrates and products across the inner membranes of mitochondria. We have localized two hitherto unidentified family members, Odc1p and Odc2p, to the inner membranes of mitochondria. They are isoforms with 61% sequence identity and we have shown in reconstituted liposomes that they transport the oxodicarboxylates 2-oxoadipate and 2-oxoglutarate by a strict counter-exchange mechanism. Intraliposomal adipate and glutarate and to a lesser extent malate and citrate supported [¹⁴C]oxoglutarate uptake. The expression of Odc1p, the more abundant isoform, made in the presence of non-fermentable carbon sources, is repressed by glucose. We consider that the main physiological roles of Odc1p and Odc2p is to supply 2-oxoadipate and 2-oxoglutarate from the mitochondrial matrix to the cytosol where they are used in the biosynthesis of lysine and glutamate respectively, and in lysine catabolism.

Thus, manipulation of the Odc1p and Odc2p transport proteins may be used to enhance the biosynthesis of lysine in yeasts, providing, for example, a lysine enriched source of single cell protein for animal feeds.

Accordingly, the present invention provides a method for modulating transport of a C5-C7 dioxocarboxylate across the mitochondrial membrane of a yeast which method comprises modulating in said yeast the activity of one or more yeast mitochondrial transport polypeptides selected from:

(a) polypeptides comprising the amino acid sequence shown as SEQ ID NO: 1 or homologues thereof; and

(b) polypeptides comprising the amino acid sequence shown as SEQ ID NO:2 or homologues thereof.

Preferably, the method of the invention comprises expressing in said yeast, one or more nucleotide sequences encoding

(a) a polypeptide having the amino acid sequence shown as SEQ ID NO: 1 or homologues thereof; and/or

(b) a polypeptide having the amino acid sequence shown as SEQ ID NO:2 or homologues thereof; and/or

(c) a fragment of the polypeptide of (a) or (b) having substantially the same activity.

Typically said C5-C7 dioxocarboxylate is selected from 2-oxoglutarate and/or 2-oxoadipate

The present invention also provides a method for increasing the rate of lysine biosynthesis in a yeast which method comprises modulating in said yeast the activity of one or more yeast mitochondrial transport polypeptides selected from:

(a) polypeptides comprising the amino acid sequence shown as SEQ ID NO:1 or homologues thereof; and

(b) polypeptides comprising the amino acid sequence shown as SEQ ID NO:2 or homologues thereof.

The present invention further provides a method of producing a foodstuff which method comprises introducing into said foodstuff a yeast or product thereof which yeast has been modified by the above method of the invention.

The present invention also provides the use of a yeast comprising a heterologous polypeptide which directs expression of one or more polypeptides selected from

(a) polypeptides having the amino acid sequence shown as SEQ ID NO:1 or homologues thereof;

(b) polypeptides having the amino acid sequence shown as SEQ ID NO:2 or homologues thereof; and

(c) fragments of the polypeptide of (a) or (b) having substantially the same activity; in the manufacture of a foodstuff.

The present invention further provides a yeast produced by the methods of the invention and a foodstuff produced by the methods of the invention.

### DETAILED DESCRIPTION OF THE INVENTION

Although in general the techniques mentioned herein are well known in the art, reference may be made in particular to Sambrook et al., Molecular Cloning, A Laboratory Manual (1989) and Ausubel et al., Short Protocols in Molecular Biology (1999) 4^{th} Ed, John Wiley & Sons, Inc (as well as the complete version Current Protocols in Molecular Biology).

A. Methods of Modulating Transport of C5-C7 dioxocarboxylates Such as 2-oxoglutarate and/or 2-oxoadipate Across the Mitochondrial Membrane of a Yeast

We have shown that the hitherto unidentified membrane proteins Odc1p and Odc2p localise to the inner membranes of mitochondria where they transport the oxodicarboxylates 2-oxodipate and 2-oxoglutarate by a strict counter-exchange mechanism. The present invention provides methods of modulating this transport process by modulating the activity of Odc1p and Odc2p. One approach for achieving this is to regulate the levels of Odc1p and/or Odc2p proteins in the mitochondrial membrane of a yeast by modulating the expression of Odc1p and/or Odc2p. For example, Odc1p and/or Odc2p proteins levels may be upregulated by introducing one or more heterologous nucleotides that direct expression of the Odc1p and/or Odc2p proteins (see below). Alternatively, or in addition, the endogenous Odc1p and/or Odc2p genes may be modified by homologous recombination to alter the levels of expression of the corresponding polypeptides (either up or down as required) using techniques well-known in the art of yeast genetics. Expression of Odc1p and/or Odc2p may also be down-regulated using anti-sense technology. Anti-sense constructs may be produced that target coding regions and/or non-coding regions of Odc1p and/or Odc2p transcripts.

Another approach, typically used to down-regulate Ode1p and/or Odc2p activity, would be to introduce compounds that inhibit Odc1p and/or Odc2p polypeptides. A number of compounds have been identified previously that inhibit specifically mitochondrial transport proteins. Suitable compounds that inhibit the transport activity of Odc1p and/or Odc2p polypeptides may be identified using in vitro assays, such as the reconstituted lipid vesicle assay described in the examples. Candidate compounds for screening in such assays include structural analogues of C5-C7 dioxocarboxylates (such as structural analogues of 2-oxodipate and/or 2-oxoglutarate). Other candidate inhibitors included truncated or mutated Odc1p/Odc2p polypeptides that bind oxodicarboxylates but fail to transport them across the mitochondrial membrane.

These approaches may be applied to any yeast which naturally expresses an Odc 1p and/or Odc2p protein, for example Saccharomyces cerevisiae.

B. Odc1p and Odc2p polypeptides/polynucleotides

The amino acid sequence of Odc1p and Odc2p polypeptides which we have identified in S. cerevisiae are shown as SEQ ID Nos 1 and 2. However, since the invention is applicable to other cells which naturally express an Odc1p and/or Odc2p protein or homologue thereof which transport 2-oxodipate and/or 2-oxoglutarate, it will be understood that Odc1p and Odc2p polypeptide sequences for use in the methods of the invention are not limited to the particular amino acid sequences shown in SEQ ID Nos 1 and 2 or fragments thereof but also include homologous sequences obtained from any source, typically other yeasts.

Thus, the present invention encompasses the use of variants, homologues or derivatives of the amino acid sequences of SEQ ID Nos 1 and 2, as well as variants, homologues or derivatives of the amino acid sequences coded for by the nucleotide sequences shown in SEQ ID Nos 1 and 2.

In the context of the present invention, a homologous sequence is taken to include an amino acid sequence which is at least 50, 60, 70, 80 or 90% identical, preferably at least 95 or 98% identical at the amino acid level over at least 50, 100 or 200 amino acids with the amino acid sequences of SEQ ID Nos 1 or 2. In particular, homology should typically be considered with respect to those regions of the sequence essential for oxodicarboxylate transport rather than non-essential neighbouring regions. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence directly compared with the corresponding amino acid in the other sequence, one residue at a time. This is called an “ungapped” alignment. Typically, such ungapped alignments are performed only over a relatively short number of residues (for example less than 50 contiguous amino acids). However, most sequence comparison methods are designed to produce optimal alignments that take into consideration possible insertions and deletions without penalising unduly the overall homology score. This is achieved by inserting “gaps” in the sequence alignment to try to maximise local homology. Common algorithms used to carry out sequence comparisons and calculate homology are implemented in software such as the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid - Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However it is preferred to use the GCG Bestfit program, typically with the default matrix and gap penalties.

Homologous polypeptides may be obtained, for example by cloning the corresponding nucleotides sequences using a variety of well-known techniques. For example, probes comprising all or part of SEQ I.D. Nos 1 or 2 may be used to probe DNA libraries made from other yeasts under conditions of medium to high stringency. Such techniques may also be used to obtain allelic variants.

Variants and strain/species homologues may also be obtained using degenerate PCR which will use primers designed to target sequences within the variants and homologues, often encoding conserved amino acid sequences within Odc1p and Odc2p sequences. Conserved sequences can be predicted, for example, by aligning the amino acid sequences from several variants/homologues. Sequence alignments can be performed using computer software known in the art. For example the GCG Wisconsin PileUp program is widely used.

The primers used in degenerate PCR will contain one or more degenerate positions and will be used at stringency conditions lower than those used for cloning sequences with single sequence primers against known sequences. It will be appreciated by the skilled person that overall nucleotide homology between sequences from distantly related organisms is likely to be very low and thus in these situations degenerate PCR may be the method of choice rather than screening libraries with labelled fragments of SEQ I.D. Nos. 1 or 2.

Alternatively, such polynucleotides may be obtained by site directed mutagenesis of characterised sequences, such as SEQ ID. Nos 1 and 2. This may be useful where for example silent codon changes are required to sequences to optimise codon preferences for a particular host cell in which the polynucleotide sequences are being expressed. Other sequence changes may be desired in order to introduce restriction enzyme recognition sites, or to alter the property or function of the polypeptides encoded by the polynucleotides.

The terms “variant” or “derivative” in relation to the Odc1p and Odc2p amino acid sequences for use in the present invention includes any substitution of, variation of, modification of, replacement of, deletion of or addition of one (or more) amino acids from or to the sequence providing the resultant amino acid sequence preferably has the ability to transport oxodicarboxylates (2-oxoadipate or 2-oxoglutarare), preferably having at least 25 to 50% of the activity as the polypeptides presented in the sequence listings, more preferably at least substantially the same activity. This may be tested, for example, by reconstituting recombinantly produced proteins into liposomes and determining transport of labelled oxoglutarate as described in the examples.

Thus Odc1p and Odc2p sequences may be modified for use in the present invention. Typically, modifications are made that maintain the transport activity of the sequence. Thus, in one embodiment, amino acid substitutions may be made, for example from 1, 2 or 3 to 10, 20 or 30 substitutions provided that the modified sequence retains at least about 25 to 50% of, or substantially the same transport activity as the sequences shown in SEQ ID Nos 1 or 2. As mentioned above, this may be tested, for example, by reconstituting recombinantly produced proteins into liposomes and determining transport of labelled oxoglutarate as described in the examples.

However, in an alternative embodiment, modifications to the amino acid sequences of a Odc1p and Odc2p polypeptide may be made intentionally to reduce the biological activity of the polypeptide. For example truncated polypeptides that bind oxodicarboxylates but fail to transport them across the mitochondrial membrane may be useful as inhibitors of the biological activity of the full length molecule.

In general, preferably less than 20%, 10% or 5% of the amino acid residues of a variant or derivative are altered as compared with the corresponding region depicted in the sequence listings.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

Polypeptides of the invention also include fragments of the above mentioned full length polypeptides and variants thereof, including fragments of the sequences set out in SEQ ID Nos 1 and 2. Suitable fragments will typically be at least about 100, 150 or 200 amino acids in length and retain the ability to transport 2-oxoadipate and/or 2-oxoglutarate across the mitochondrial membrane. Polypeptide fragments of the Odc1p and Odc2p proteins and allelic and species variants thereof may contain one or more (e.g. 2, 3, 5, or 10) substitutions, deletions or insertions, including conserved substitutions. Where substitutions, deletion and/or insertions have been made, for example by means of recombinant technology, preferably less than 20%, 10% or 5% of the amino acid residues depicted in the sequence listings are altered.

The Odc1p and Odc2p proteins for use in the present invention are typically made in vivo by recombinant means as described below. Since Odc1p and Odc2p proteins have been shown herein to be located in the inner mitochondrial membrane, generally, Odc1p and Odc2p proteins and nucleotides encoding the same will contain targeting sequences to ensure that the proteins are expressed and targeted to the correct location in the inner mitochondrial membrane. The native Odc1p/Odc2p mitochondrial signal sequences may be used. Alternatively, other suitable mitochondrial signal sequences may be used.

Polynucleotides for use in the invention comprise nucleic acid sequences encoding Odc1p or Odc2p amino acid sequences as described above. It will be understood by a skilled person that numerous different polynucleotides can encode the same polypeptide as a result of the degeneracy of the genetic code. In addition, it is to be understood that skilled persons may, using routine techniques, make nucleotide substitutions that do not affect the polypeptide sequence encoded by the polynucleotides of the invention to reflect the codon usage of any particular host organism in which the polypeptides of the invention are to be expressed.

Odc1p and Odc2p polynucleotides for use in the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the present invention, it is to be understood that the polynucleotides described herein may be modified by any method available in the art. Such modifications may be carried out in order to enhance the in vivo activity or life span of the polynucleotides.

The terms “variant”, “homologue” or “derivative” in relation to the ODC1 and ODC2 nucleotide sequences for use in the present invention include any substitution of, variation of, modification of, replacement of, deletion of or addition of one (or more) nucleic acid from or to the sequence providing the resultant nucleotide sequence codes for a polypeptide having Odc1p or Odc2p transport activity, preferably having at least the same activity as the polypeptide sequences presented in the sequence listings.

As indicated above, with respect to sequence homology, preferably there is at least 75%, more preferably at least 85%, more preferably at least 90% homology to the sequences shown in the sequence listing herein. More preferably there is at least 95%, more preferably at least 98% homology. Nucleotide homology comparisons may be conducted as described above. A preferred sequence comparison program is the GCG Wisconsin Bestfit program described above, using the default parameters.

Also suitable for use in the present invention are nucleotide sequences that are capable of hybridising selectively to the sequences presented herein, or any variant, fragment or derivative thereof, or to the complement of any of the above. Nucleotide sequences are preferably at least 300 nucleotides in length, more preferably at least 450, 600 or 750 nucleotides in length.

The term “selectively hybridizable” means that the polynucleotide used as a probe based on the nucleotides sequences shown in the sequence listings is used under conditions where a target Odc1p/Odc2p polynucleotide is found to hybridize to the probe at a level significantly above background conditions (e.g. 65° C. and 0.1×SSC {1×SSC=0.15 M NaCl, 0.015 M Na₃ Citrate pH 7.0}). The background hybridization may occur because of other polynucleotides present, for example, in the cDNA or genomic DNA library being screening. In this event, background implies a level of signal generated by interaction between the probe and a non-specific DNA member of the library which is less than 10 fold, preferably less than 100 fold as intense as the specific interaction observed with the target DNA. The intensity of interaction may be measured, for example, by radiolabelling the probe, e.g. with ³²P.

C. Expression Vectors

Typically, a polynucleotide encoding a Odc1p or Odc2p polypeptide is part of a vector where it is operably linked to a regulatory control sequence which is capable of providing for the expression of the coding sequence by the host cell, i.e. the vector is an expression vector. The term “operably linked” refers to a juxtaposition wherein the components described are in a relationship permitting them to function in their intended manner. A regulatory sequence “operably linked” to a coding sequence is ligated in such a way that expression of the coding sequence is achieved under condition compatible with the control sequences. Control sequences operably linked to sequences encoding the protein of the invention include promoters/enhancers and other expression regulation signals. These control sequences may be selected to be compatible with the host cell for which the expression vector is designed to be used in. The term “promoter” is well-known in the art and encompasses nucleic acid regions ranging in size and complexity from minimal promoters to promoters including upstream elements and enhancers.

Aside from the promoter native to the genes encoding the Odc1p and Odc2p polypeptides, other promoters may be used to direct expression of the Odc1p and Odc2p polypeptides. The promoter may be selected for its efficiency in directing the expression of these polypeptides in the desired expression host.

Regulatory sequences may be inducible or regulated such that expression of the polypeptides only takes place in response to certain stimuli or conditions. Alternatively, a constitutive promoter may be selected to direct the expression of the Odc1p and/or Odc2p polypeptides. Examples of strong yeast promoters are those obtainable from the genes for alcohol dehydrogenase, lactase, 3-phosphoglycerate kinase and triosephosphate isomerase. Hybrid promoters may also be used to improve inducible regulation of the expression construct.

Such vectors may be transformed into a suitable host cell to provide for expression of Odc1p and/or Odc2p protein. Suitable host cells include yeast cells, such as yeast cells of the genus Kluyveromyces or Saccharomyces.

The vectors may be for example, plasmid or virus vectors provided with an origin of replication, optionally a promoter for the expression of the said polynucleotide and optionally a regulator of the promoter. The vectors may contain one or more selectable marker genes. The most suitable selection systems for industrial micro-organisms are those formed by the group of selection markers which do not require a mutation in the host organism.

According to one embodiment of the present invention where, for example, it is desired to produce yeast cultures comprising increased levels of lysine for use in animal feeds, the production of the Odcp1 and/or Odc2p polypeptides can be effected by the culturing of microbial expression hosts, which have been transformed with one or more polynucleotides of the present invention, in a conventional nutrient fermentation medium.

The fermentation medium can comprise a known culture medium containing a carbon source (e.g. glucose, maltose, molasses, etc.), a nitrogen source (e.g. ammonium sulphate, ammonium nitrate, ammonium chloride, etc.), an organic nitrogen source (e.g. yeast extract, malt extract, peptone, etc.) and inorganic nutrient sources (e.g. phosphate. magnesium, potassium, zinc, iron, etc.).

The selection of the appropriate medium may be based on the choice of expression hosts and/or based on the regulatory requirements of the expression construct. Such media are well-known to those skilled in the art. The medium may, if desired. contain additional components favouring the transformed expression hosts over other potentially contaminating microorganisms.

Alternatively, in a particularly convenient embodiment, nutrient-rich waste, such as cheese whey may be used as a growth medium.

The fermentation can be preformed over a period of 0.5-20 days in a batch or fed-batch process suitably at a temperature in the range of between 0 and 45° C. and, for example, a pH between 2 and 10. Preferred fermentation conditions are a temperature in the range of between 20 and 37° C. and/or a pH between 3 and 9. The appropriate conditions are usually selected based on the choice of the expression host

After fermentation, the cells can be removed from the fermentation broth by means of centrifugation or filtration. The cells may then be used to produce animal feed using standard processing procedures.

In another embodiment, the fermentation medium may be a foodstuff such as a bakery product or a precursor thereof, such as dough. The transformed yeast host cells may be introduced into the foodstuff or precursor thereof in the normal manner (e.g. as in the introduction of S. cerevisiae into dough during the bread making process). Typically, the yeast cells remain part of the foodstuff through to the final product.

D. Uses

The methods of the present invention may be used to manipulate aspects of cellular metabolism in yeasts by altering the kinetics of biochemical pathways in which the C5-C7 oxodicarboxylates such as 2-oxoadipate and 2-oxoglutarate are intermediates as a result of an increase or decrease in the rate of transport of these compounds across the mitochondrial membrane. For example, an increase in the activity of Odc1p and/or Odc2p may increase the concentration of 2-oxoadipate in the cytoplasm resulting in an increase in the biosynthesis of lysine (and glutamate).

It may be desirable to further manipulate other proteins involved in lysine biosynthesis to reduce the possibility that negative feedback control mechanisms will act to limit lysine accumulation.

Yeasts that have been manipulated to increase their rate of lysine biosynthesis may be used in the production of animal feed supplements and in human food production. For example, S. cerevisiae strains produced by the methods of the invention that have increased lysine production may be used in bakery products to increase their lysine content. K lactis strains produced by the methods of the invention may be used to ferment waste whey to produce lysine-enriched protein products for use in animal feed. Alternatively, yeast strains produced by the methods of the invention may be grown in normal medium under aerobic or anaerobic conditions to produce lysine-enriched yeast biomass for addition to human or animal food products.

The invention will now be further described by way of Examples, which are meant to serve to assist one of ordinary skill in the art in carrying out the invention and are not intended in any way to limit the scope of the invention. The Examples refer to the Figures. In the Figures:

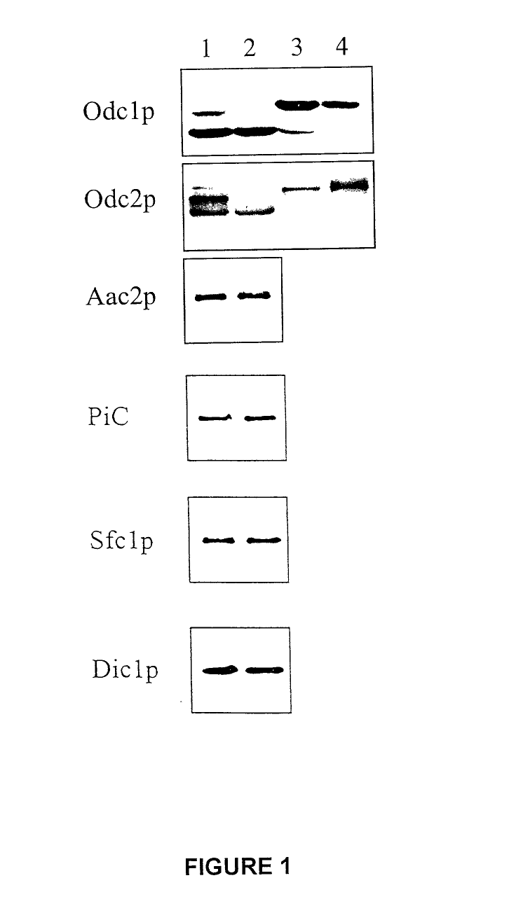

FIG. 1 Immunoblot analysis of ODC proteins in yeast mitochondria. 25 μg mitochondrial protein from WT (lane 1) and odc1Δodc2Δ cells (lane 2) were separated by SDS-PAGE, transferred to nitrocellulose and immunodecorated with antibodies directed against Odc1p, Odc2p and the ADP/ATP carrier (Aac2p). Lane 3: 10 μg and 1 μg of mitochondrial protein from odc1Δodc2Δ/pODC1 and odc1 Δodc2Δ/pODC2 cells, respectively. Lane 4: 350 ng (Odc1p) and 5 ng (Odc2p) of recombinant ODC transport proteins purified from mitochondria in lane 3.

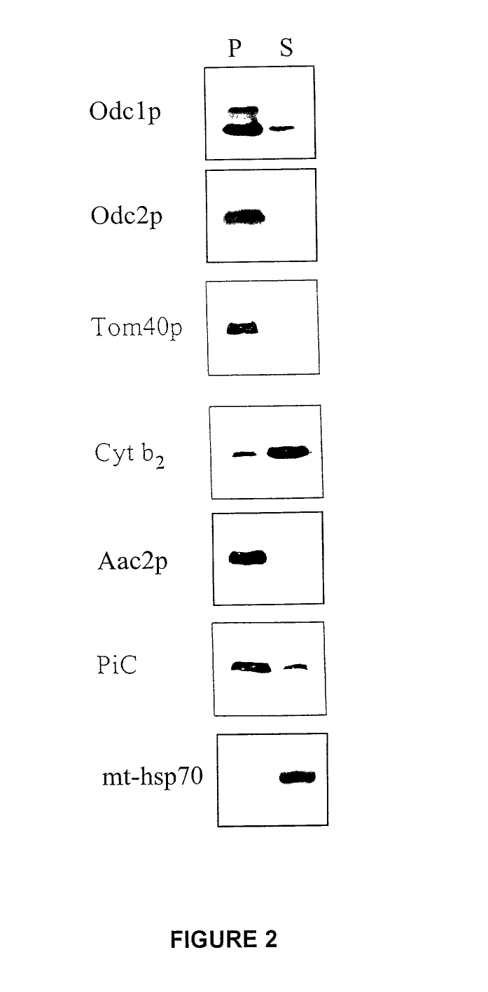

FIG. 2. Submitochondrial localization of Odc1p and Odc2p. Analysis by SDS-PAGE and Western blotting of (S), soluble and peripheral proteins, and (P), intrinsic membrane proteins from yeast mitochondria blotted with antisera directed against Odc 1p, Odc2p, the ADP/ATP carrier (Aac2p; inner membrane component) and the mitochondrial hsp70 (mt-hsp70; matrix protein).

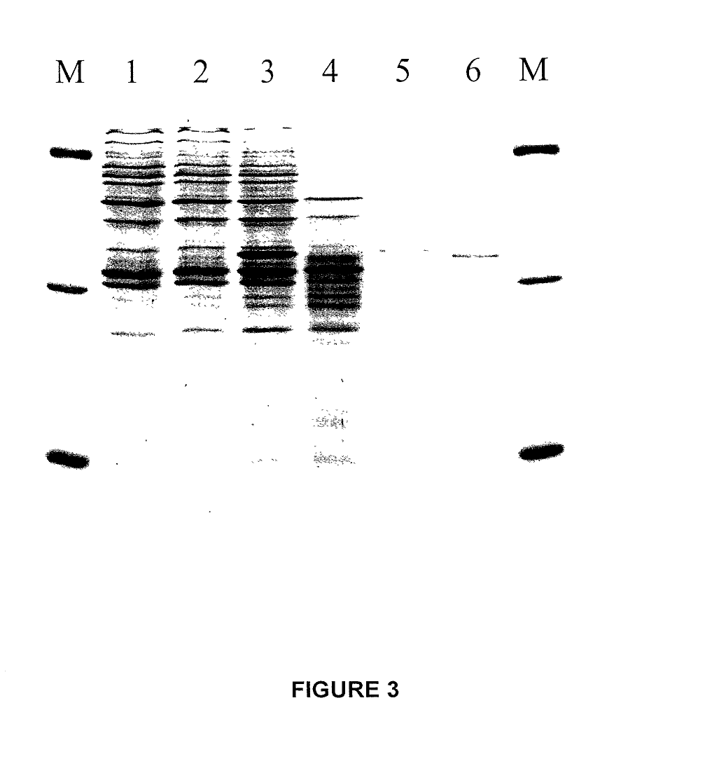

FIG. 3. Purification of the over-expressed ODC proteins. Proteins were separated by SDS-PAGE and stained with Coomassie-blue dye. Lanes M, markers (bovine serum albumin, carbonic anhydrase and cytochrome c). Lanes 1-4, mitochondrial protein (100 μg) from wild-type (lane 1), odc1Δ odc2Δ mutant (lane 2), odc1Δodc2Δ/pODC1 (lane 3) and odc1Δodc2Δ/pODC2 (lane 4) strains. Cells were harvested 6 h after addition of galactose. Lanes 5 and 6, 2 μg Odc1p (lane 5) and 4 μg Odc2p (lane 6) purified from mitochondria in lanes 3 and 4 respectively.

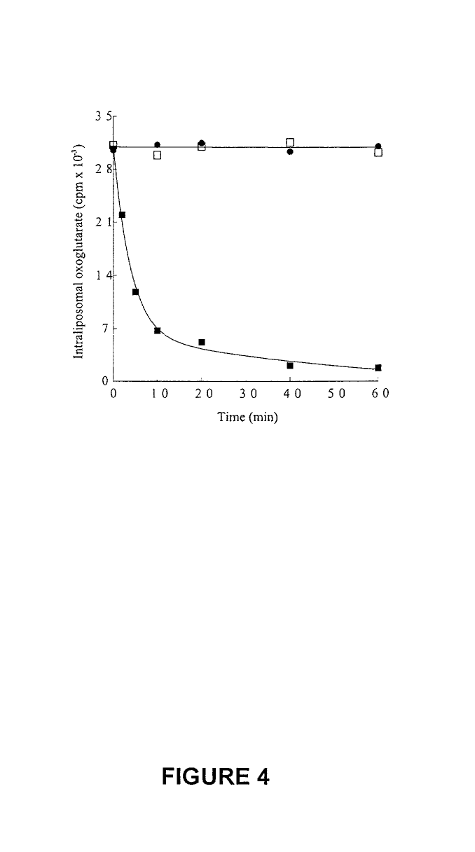

FIG. 4. Efflux of [¹⁴C]oxoglutarate from proteoliposomes. Proteoliposomes were reconstituted with recombinant Odc1p in the presence of 20 mM oxoglutarate, and then the internal substrate pool was labelled by carrier-mediated exchange equilibration. After removal of external substrate by Sephadex G-75 chromatography, the efflux of [¹⁴C]oxoglutarate was started by adding buffer F alone (●) or 10 mM oxoglutarate (▪), or 10 mM oxoglutarate, 30 mM pyridoxal 5′-phosphate and 10 mM bathophenanthroline (□) in the same buffer.

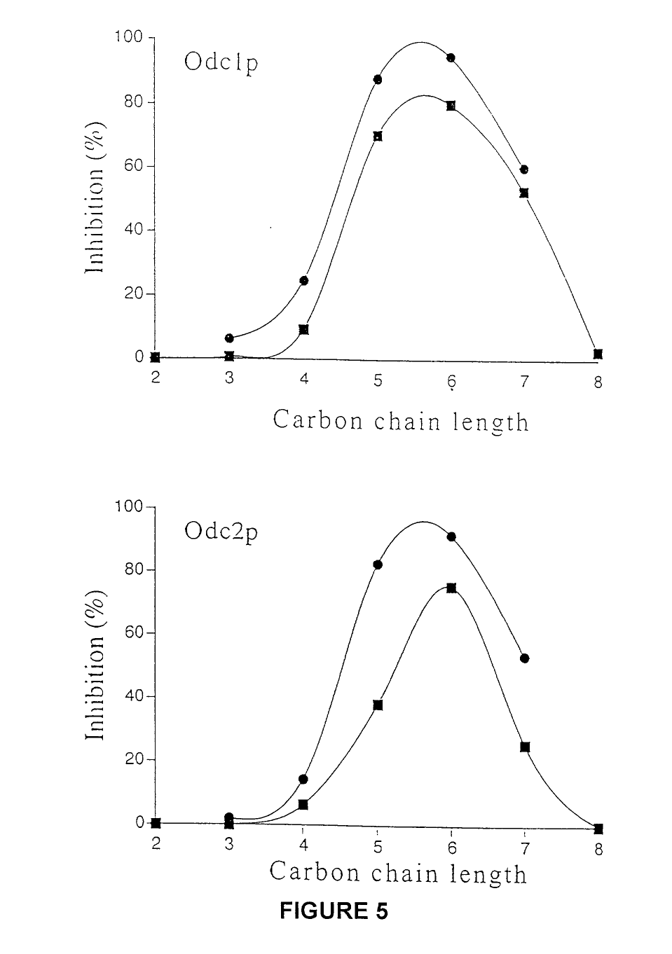

FIG. 5. Effect of dicarboxylates and 2-oxodicarboxylates with different carbon chain length on the rate of [¹⁴C]oxoglutarate uptake into proteoliposomes reconstituted with recombinant Odc1p. Proteoliposomes were preloaded with 20 mM oxoglutarate and reconstituted with recombinant Odc1p. Transport was initiated by the addition of 0.4 mM [¹⁴C]oxoglutarate and terminated after 45 seconds. Oxalate, malonate, succinate, glutarate, adipate, pimelate, suberate (▪) and the corresponding 2-oxoacids (●) were added simultaneously with [1⁴C]oxoglutarate at 4.0 mM concentration. The control value for uninhibited 2-oxoglutarate uptake was 108 mmol/min per g protein. The results are given as percentage inhibition of the control. Similar results were obtained in three separate experiments in duplicate.

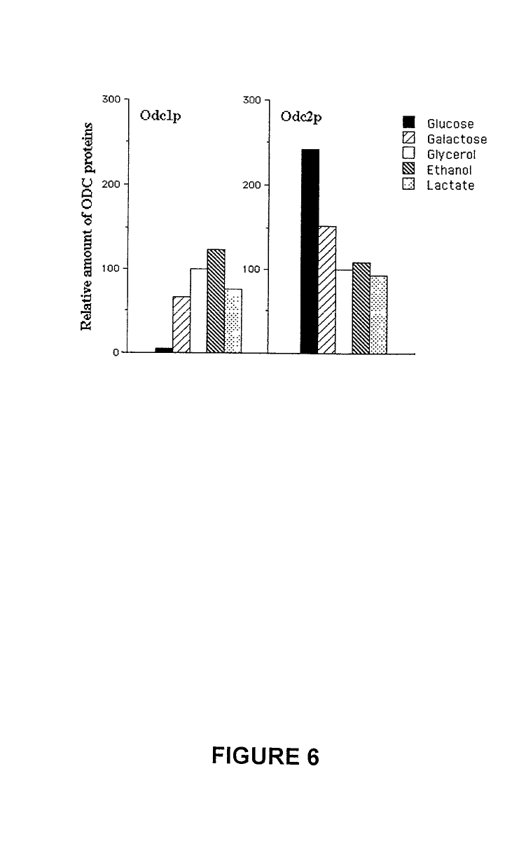

FIG. 6. Comparison of the expression of Odc1p and Odc2p on various carbon sources. Cells were harvested from exponentially growing cells on YP medium supplemented with the indicated carbon sources. Amounts of ODC proteins were estimated by densitometry upon immunodecoration of mitochondrial proteins with specific antisera. Similar results were obtained in three independent experiments in duplicate. The amount of Odc1p and Odc2p present in mitochondria from glycerol-fed cells was taken as 100%.

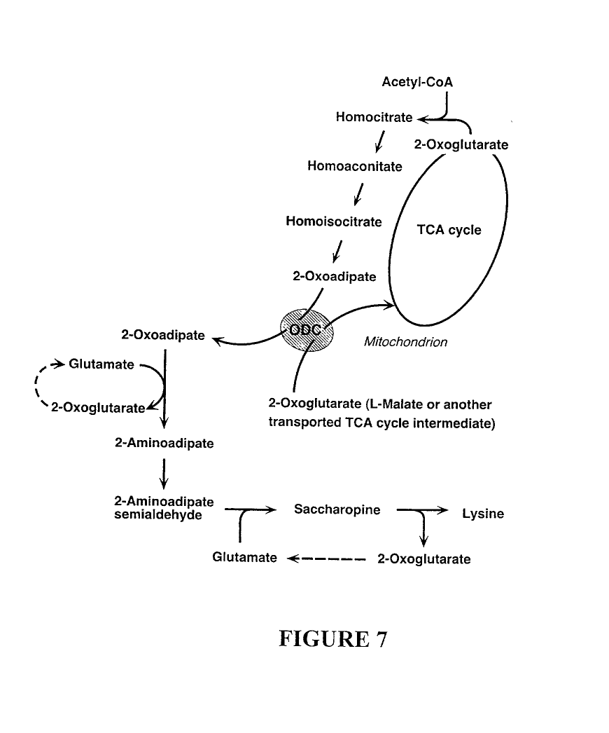

FIG. 7. Compartmentalizat of selected enzymes involved in lysine biosynthesis in S. cerevisiae and the role of the mitochondrial oxodicarboxylate carrier (ODC). The dashed lines indicate the entry of nitrogen into the glutamate molecule.

### EXAMPLES

### Materials and Methods

### Yeast Strains and Growth Conditions

Deletion of the yeast nuclear genes ODC1 (ORF YPL134c) and ODC2 (ORF YOR222w) was accomplished by sequential homologous recombination of the auxotrophic markers TRPI and HIS3 at the respective loci of Saccharomyces cerevisiae YPH499 strain (wild type: MATa ade2-101 his3-A200 leu2-Δ1 ura3-52 trpl-A63 lys2-801). Deletants were verified by PCR and western-blot analysis. Yeast cells were precultured on synthetic complete (SC) medium (8) supplemented with 3% glycerol and 0.1% glucose. Tryptophan, histidine and uracil were omitted where the genotype permitted. For growth studies, exponentially growing cells were harvested by centrifugation, washed with growth medium and diluted with the same medium until a final optical density of 5×10³ at 600 nm was reached. For the preparation of mitochondria, precultures were diluted 35-fold in YP medium (1% yeast extract, 2% bacto-peptone, pH adjusted to 4.8 with HCl) and grown in the presence of the same carbon sources to mid exponential phase. Galactose (0.45%) was added 6 h before harvesting. For the estimation of the expression of ODCs, yeast cells were grown at 30° C. to mid-log phase in YP medium supplemented with either 2% glucose, 2% galactose, 3% glycerol, 2% ethanol or 3% lactate and then harvested by centrifugation (3000×g, 5 min).

### Sub-fractionation of Mitochondria and Quantitative Immunoblotting

Extractions of mitochondria with sodium carbonate or with digitonin were performed as described previously (6). To determine the amount of Odc1p and Odc2p in wild-type mitochondria, standard calibration curves were constructed using 10-500 ng pure recombinant ODC proteins as standards. After transfer of the proteins to the same nitrocellulose membrane, the standards and the mitochondrial samples were immunodecorated simultaneously. Once it had been verified that the sample loading was within the linear range of the calibration curves, the densitometric signal intensity was used to measure the amount of Odc1p and Odc2p.

### Construction of the ODC Expression Plasmids

The coding sequences of ODC1 and ODC2 were amplified from S. cerevisiae genomic DNA by PCR. Forward and reverse oligonucleotide primers were synthesized corresponding to the extremities of the ODC sequences with additional HindIII and BamHI sites, respectively. The reverse primers also contained 18 additional bases encoding a 6-histidine tag immediately before the translational termination codon. The products of PCR were cloned into the expression vector pYES2 (Invitrogen, Groningen, The Netherlands). The resulting expression plasmids (pODC1 or pODC2) were introduced in the odc1Δodc2Δ double mutant, and transformants (odc1Δodc2Δ/pODC1 or odc1 Δodc2Δ/pODC2 cells) were selected for uracil auxotrophy. Other experimental conditions have been described before (9).

### Over-expression in S. cerevisiae and Purification of the ODC Proteins

Mitochondria were isolated from odc1Δodc2Δ/pODC1 or odc1Δodc2Δ/pODC2 cells according to standard procedures (10) and solubilized in buffer A (500 mM NaCl, 10 mM PIPES, pH 7.0) containing 0.8% digitonin (w/v) and 0.1 mM PMSF (phenylmethylsulfonyl fluoride), at a final concentration of 0.2-0.4 mg protein/ml. After incubation for 20 min at 4° C., the mixture was centrifuged (138000×g, 20 min). The supernatant (1.1 ml) was mixed for 1 hour at 4° C. with 0.45 ml Ni-NTA agarose (Qiagen, Hilden, Germany) previously equilibrated with buffer A. Then the resin was packed into a column (0.5 cm internal diameter) and washed extensively with the following buffers: B, 500 mM NaCl, 0.8% digitonin, 10 mM imidazole, 0.5% Triton X-100. 7.5% glycerol, 10 mM PIPES, pH 7.5 (2 ml); C, 300 mM NaCl, 0.8% digitonin, 10 mM imidazole, 0.1% Triton X-100, 5% glycerol, 10 mM PIPES, pH 7.5 (2 ml); D, 100 mM NaCl, 0.6% digitonin, 10 mM imidazole, 0.05% Triton X-100, 1% glycerol, 10 mM PIPES, pH 7.5 (1 ml); E, 50 mM NaCl, 0.3% digitonin, 10 mM imidazole, glycerol 0.5%, 10 mM PIPES, pH 7.0 (1 ml). Finally pure ODC proteins were eluted with a buffer containing 50 mM NaCl, 0.1% digitonin, 80 mM imidazole and 10 mM PIPES, pH 7.0. Protein concentrations were determined by the Lowry method modified for the presence of detergent (11) or by laser densitometry (9).

### Protein Chemical Characterization of Over-expressed ODC Isoforms

Proteins were analysed by SDS-PAGE in 17.5% gels (12) and either stained with Coomassie blue dye or transferred to nitrocellulose membranes. The identities of purified Odc1p and Odc2p were confirmed by matrix-assisted laser desorption ionization-time of flight (MALDI-TOF) mass spectrometry of trypsin digests of the corresponding bands excised from a Coomassie-stained gel. Western blotting was carried out with rabbit antibodies against the bacterially expressed ODC proteins. The overproduction of ODC isoforms as inclusion bodies in the bacterial cytosol and the purification of the inclusion bodies in host strain E. coli C0214(DE3) have been described previously (9). The films were scanned with an LKB 2202 Ultroscan laser densitometer.

Reconstitution of the ODC proteins into liposomes—Purified ODC proteins were reconstituted by cyclic removal of the detergent with a hydrophobic column (13). The composition of the initial mixture used for reconstitution was: 200 μl of purified isoform (0.3-0.4 μg of protein), 70 μl of 10% Triton X-114, 100 μl of 10% phospholipids in the form of sonicated liposomes, 20 mM oxoglutarate (except where otherwise indicated), 10 mM Pipes (pH 7.0), 0.7 mg of cardiolipin (Sigma) and water to a final volume of 700 μl. These components were mixed thoroughly, and the mixture was recycled 13 times through an Amberlite column (Supelco) (3.0 cm×0.5 cm) preequilibrated with a buffer containing 10 mM Pipes (pH 7.0) and with the substrate at the same concentration as in the starting mixture. All operations were performed at 4° C., except the passages through Amberlite, which were carried out at room temperature.

### Transport Measurements

External substrate was removed from proteoliposomes on a Sephadex G-75 column preequilibrated with buffer F (50 mM NaCl and 10 mM PIPES, pH 7.0). Transport at 25° C. was started by adding [¹⁴C]oxoglutarate (unless otherwise indicated) to the proteoliposomes, and terminated by addition of 30 mM pyridoxal 5′-phosphate and 10 mM bathophenanthroline (the “inhibitor-stop” method (13)). In controls, inhibitors were added with the labelled substrate. The external radioactivity was removed on Sephadex G-75 and the internal radioactivity was measured. The transport activity was the difference between experimental and control values. The initial rate of transport was calculated in mmol/min per g protein from the time course of isotope equilibration (13). Various other transport activities were also assayed by the inhibitor-stop method. For efflux measurements, the internal substrate pool of the proteoliposomes was made radioactive by carrier-mediated exchange equilibration (13) with 0.1 mM [¹⁴C]oxoglutarate added at high specific radioactivity. After 60 min, the residual external radioactivity was removed by passing the proteoliposomes again through a column of Sephadex G-75. Efflux was started by adding unlabelled external substrate or buffer F alone and terminated by adding the inhibitors indicated above.

### Results

### Subcellular Localization of the ODC Proteins

Immunoreactive bands on SDS-PAGE gels were detected with antibodies against Odc1p and Odc2p. Bands with apparent molecular masses of about 36.5 and 35.0 kDa, respectively, were detected in wild-type mitochondria (FIG. 1, lane 1) but not in mitochondria from the odc1Δodc2Δ double mutant (FIG. 1, lane 2). The antibody against Odc2p cross-reacted with Odc 1p (upper band) and both antibodies reacted with an unidentified band of about 33.0 kDa which was also present in the odc1Δodc2Δ mitochondria. The contents of the ADP/ATP carrier and (not shown) the phosphate. succinate-fumarate and dicarboxylate carriers detected with specific antibodies were essentially the same in both wild-type and odc1Δodc2Δ mitochondria. Therefore, the absence of both ODC proteins from the double mutant does not affect the expression of other mitochondrial carriers. Furthermore, the phenotype of the odc1Δodc2Δ strain was studied by comparison of the growth of the mutant cell with the parental strain in shake-flask cultures on different media. Both the wild-type and the deletion strain yeast exhibited substantial and similar growth on either rich medium (YP) or synthetic medium (SC) supplemented with either 2% glucose. 2% galactose, 3% glycerol, 2% ethanol or 3% lactate, indicating that the absence of the ODC proteins does not impair the respiratory function of mitochondria.

The submitochondrial location of Odc1p and Odc2p was examined by separation of soluble and peripheral proteins from integral membrane proteins of wild-type mitochondria by carbonate treatment (FIG. 2). Both Odc1p and Odc2p remained in the membrane protein fraction, as did the ADP/ATP carrier and (not shown) Tom40p (marker proteins of inner and outer mitochondrial membranes, respectively), but the matrix protein hsp70 and (not shown) the intermembrane space protein cytochrome b₂ were in the soluble and peripheral protein fraction. In other experiments, at 0.3% digitonin more than 80% of the outer membrane protein Tom40p, and less than 10% of Odc1p, Odc2p and the ADP/ATP carrier, were solubilized from wild-type mitochondria. At higher levels of digitonin, Odc1p and Odc2p were solubilized progressively in parallel with the ADP/ATP carrier (data not shown). Therefore, Odc1p and Odc2p are integral proteins of the inner mitochondrial membrane.

### Expression in S. cerevisiae and Purification of the ODC Proteins

Odc1p and Odc2p were overexpressed at high levels in a S. cerevisiae strain devoid of both corresponding genes (odc1Δodc2Δ strain) (FIG. 3, lanes 3 and 4). Their apparent molecular masses were about 38 and 37 kDa (the calculated values including the initiator methionine and the histidine tail were 35006 and 34807 Da respectively). The successful over-expression and targeting of the episomal Odc1p and Odc2p to mitochondria was confirmed by Western blotting of isolated mitochondria from the odc1Δodc2Δ/pODC strains (FIG. 1, lane 3), since the amount of mitochondrial protein applied in lane 3 of FIG. 1 was 2.5 (from odc1Δodc2Δ/pODC1 cells) and 25 (from odc1Δodc266 /pODC2 cells) times less than the amount of wild-type mitochondrial protein applied in lane 1 of FIG. 1. The differences in the molecular mass of immunodecorated bands in lane 3 odc1Δodc2Δ/pODC mitochondria) and lane 1 (wild-type mitochondria) is a reflection of the presence of a C-terminal histidine tag in the recombinant proteins.

The presence of the histidine tail at the C-terminal end of the expressed ODC isoforms allowed their purification by a Ni⁺-agarose affinity column (FIG. 3, lanes 5-6 and FIG. 1, lane 4). About 0.7 mg of Odc1p and about 0.1 mg of Odc2p were obtained per liter of culture. The identity of purified Odc1p and Odc2p was confirmed by MALDI-TOF mass spectrometry.

### Functional Characterization of Recombinant Odc1p and Odc2p

Proteoliposomes reconstituted with digitonin-solubilized mitochondria isolated from odc1Δodc2Δ/pODC1 or odc1Δodc2Δ/pODC2 strains were able to catalyze an active [¹⁴C]oxoglutarate/oxoglutarate homoexchange (see Table 1). A lower oxoglutarate transport was observed upon reconstitution of the digitonin extract from wild-type mitochondria, whereas liposomes reconstituted with the extract from odc1Δodc2Δ mitochondria showed a very low but reproducible oxoglutarate exchange. Furthermore, oxoglutarate transport, measured upon reconstitution of the mitochondrial extract isolated from the double deletion strain transformed with the pYES2 vector harbouring the sequence encoding the yeast oxaloacetate carrier (odc1Δodc2Δ/pOAC1 strain) (6), the yeast carnitine carrier (odc1Δodc2Δ/pCRC1 strain) (7), or with the empty pYES2 vector (not shown), was not significantly increased.

### Table 1—Oxoglutarate Homoexchange in Liposomes Reconstituted with Mitochondrial Extracts from Various Yeast Strains

Proteoliposomes were preloaded internally with 20 mM oxoglutarate. Transport was started by the external addition of 0.1 mM [¹⁴C]oxoglutarate. The data represent the means ±S.D. of at least three different experiments.

| | [¹⁴C]oxoglutarate |
|---|---|
| | uptake(μmol/min per g |
| Strain | protein) |
| WT | 7.2 ± 2.0 |
| odc1Δodc2Δ | 0.8 ± 0.2 |
| odc1Δodc2Δ/pODC1 | 85.0 ± 14.9 |
| odc1Δodc2Δ/pODC2 | 26.8 ± 5.6 |
| odc1Δodc2Δ/pOAC1 | 1.4 ± 0.4 |
| odc1Δodc2Δ/pCRC1 | 1.0 ± 0.3 |

The purified and reconstituted Odc1p and Odc2p catalyzed a very active [¹⁴C]oxoglutarate/oxoglutarate exchange, which was inhibited by a mixture of pyridoxal 5′-phosphate and bathophenanthroline. No such activity was found with Odc1p and Odc2p that had been boiled before incorporation into liposomes. Likewise, no [¹⁴C]oxoglutarate uptake was observed into proteoliposomes that did not contain internal oxoglutarate, indicating that Odc1p and Odc2p do not catalyze a unidirectional transport (uniport) of oxoglutarate, but only the exchange reaction. In order to obtain further information about the mechanism of transport catalyzed by Odc1p and Odc2p, the efflux of [¹⁴C]oxoglutarate from pre-labelled active proteoliposomes was investigated as it provides a more convenient assay for unidirectional transport (13). An experiment performed with proteoliposomes reconstituted with Odc1p is shown in FIG. 4. In the absence of external substrate no efflux was observed even after incubation for 1 hour. However, upon addition of external oxoglutarate, an extensive efflux of intraliposomal radioactivity occurred and this efflux was prevented completely by the presence of the inhibitors pyridoxal 5′-phosphate and bathophenanthroline (FIG. 4). Similar data were obtained using Odc2p instead of Odc1p. These results show clearly that reconstituted Odc1p and Odc2p catalyze an obligatory exchange reaction of internal oxoglutarate for external oxoglutarate. Furthermore, the proteoliposomes did not catalyze homoexchanges for glutamate, aspartate, 2-oxoisocaproate, glutamine, ornithine, ADP, phosphate, sulfate and carnitine (internal concentration, 10 mM; external concentration, 1 mM).

The substrate specificity of purified Odc1p and Odc2p was investigated further by measuring the uptake of [¹⁴C]oxoglutarate into proteoliposomes which had been preloaded with various substrates. As shown in Table II, [¹⁴C]oxoglutarate was taken up efficiently by proteoliposomes containing internal oxoglutarate, 2-oxoadipate, 2-oxopimelate, glutarate, adipate, pimelate, L-malate and D-malate. A much lower activity was observed in the presence of internal oxaloacetate, succinate, citrate and isocitrate. A very low activity was found with internal malonate, suberate, fumarate and maleate, and (not shown) no exchange was found with oxalate, aspartate, glutamate, 2-aminoadipate, 2-aminopimelate, pyruvate, 2-oxobutyrate, 2-hydroxybutyrate, 2-oxovalerate, 2-oxoisocaproate, phosphate, sulfate, thiosulfate, ADP ATP, ornithine, glutamine, and carnitine.

The [¹⁴C]oxoglutarate/oxoglutarate exchange reactions catalyzed by reconstituted Odc1p and Odc2p were inhibited strongly by mercurials (mersalyl, p-chloromercuribenzene sulfonate and mercuric chloride), by pyridoxal 5′-phosphate, by bathophenanthroline and by α-cyanocinnamate (see Table III). Also, both Odc1p and Odc2p were inhibited considerably by N-ethylmaleimide. The impermeable dicarboxylate analogues butylmalonate and phenylsuccinate, which are known to be powerful inhibitors of the oxoglutarate and dicarboxylate carriers (14, 15), decreased the reconstituted transport activities rather poorly. Also, the tricarboxylate analogue 1,2,3-benzenetricarboxylate, a very efficient inhibitor of the citrate carrier (16), had a rather mild inhibitory effect, and carboxyatractyloside, a powerful inhibitor of the ADP/ATP carrier (17), had little or no effect on the activities of Odc1p and Odc2p.

In addition, the ability of non-radioactive potential substrates to inhibit the oxoglutarate/oxoglutarate exchange was examined. The effectiveness of dicarboxylates and 2-oxodicarboxylates with different carbon chain length on the rate of oxoglutarate uptake are compared in FIG. 5. With both reconstituted Odc 1p and (not shown) Odc2p, glutarate, adipate and pimelate with 5-7 carbon atoms caused a significant inhibition of oxoglutarate uptake, whereas oxalate, malonate, succinate and suberate had virtually no effect. The presence of a carbonyl group on the dicarboxylate molecule enhanced the inhibitory effect of the compounds with a maximum of inhibition at 6 carbon atoms. In similar experiments (not shown) the effect of other dicarboxylates on the rate of oxoglutarate uptake catalyzed by the recombinant and reconstituted Odc1p and Odc2p was also tested. The presence of a hydroxyl group on the C₄ dicarboxylate molecule (as in malate), increased about 10-fold the extent of the inhibition of oxoglutarate uptake with respect to the corresponding dicarboxylate. Also, L- and D-tartrate inhibited the uptake of oxoglutarate more efficiently than succinate, their inhibitory effect being comparable to that of malate. However, the C5 hydroxydicarboxylate, hydroxyglutarate, was slightly less effective than glutarate and the C₃ hydroxydicarboxylate (tartronate) was completely ineffective like the corresponding dicarboxylate. Both the cis- and trans-unsaturated dicarboxylates, fumarate and maleate, had no effect, nor had the aminodicarboxylates. DL-Threo-hydroxyaspartate has been reported to inhibit the uptake of oxoglutarate into intact yeast mitochondria (18), but it had no affect on oxoglutarate transport catalyzed by reconstituted Odc1p and Odc2p. In view of the inhibition of oxoglutarate uptake by dicarboxylates carrying a carbonyl group, the effect of oxomonocarboxylates on the rate of uptake of 0.4 mM oxoglutarate was also tested. Pyruvate, 2-oxovalerate 2-oxobutyrate, 2-oxoisocaproate and 5-oxohexanoate (all at 8 mM concentration, i.e. 20 times greater than the substrate), did not influence the rate of oxoglutarate uptake (data not shown). Finally, several aminomonocarboxylates, (glycine, alanine, valine, threonine and serine), and other amino acids (glutamine, asparagine, lysine, arginine, histidine and ornithine) had no effect on the oxoglutarate/oxoglutarate exchange (data not shown).

### Kinetic Characteristics of Recombinant Odc1p and Odc2p

The kinetic constants of the recombinant purified Odc1p and Odc2p were determined by measuring the initial transport rate at various external [¹⁴C]oxoglutarate concentrations, in the presence of a constant saturating internal concentration of 20 mM oxoglutarate. The transport affinities (Km) and the specific activities (Vmax) for the oxoglutarate/oxoglutarate exchange at 25° C., were 0.52±0.08 mM and 252±53 mmol/min per g protein for Ode1p (24 experiments) and 0.47±0.07 mM and 73±17 mmol/min per g protein for Odc2p (16 experiments). All of the compounds summarized in Table IV inhibited oxoglutarate uptake by both isoforms competitively, because they were found to increase the apparent Km without changing the Vmax of oxoglutarate uptake (not shown). The inhibition constants (Ki) of 2-oxoadipate are only about 2-fold lower than the Km of oxoglutarate, but about 5-7 times lower than the Ki values of malate and 2-oxopimelate, and more than 50 times lower than those of oxaloacetate and succinate. The Ki values of malate and citrate are similar to the Km values of the same substrates for the reconstituted Odc1p, as determined from Lineweaver-Burk plots of the rate of [¹⁴C]malate or [¹⁴C]citrate uptake in the presence of a constant internal oxoglutarate concentration of 20 mM. Under these conditions the Km of malate was 1.3 ±0.2 mM (7 experiments) and that of citrate 5.7±0.5 mM (3 experiments). Taken together these results demonstrate that 2-oxoadipate and 2-oxoglutarate are the best substrates for reconstituted Odc1p and Odc2p.

### Influence of the Carbon Source on the Expression of ODC Proteins

As Odc1p and Odc2p appear to have virtually the same transport properties, in order to shed light on the metabolic significance of the ODC isoforms the regulation of protein expression was examined. To quantify Odc1p and Odc2p, various amounts of mitochondrial samples from yeast cells fed on glycerol were loaded onto the gel and immunoblotted simultaneously with the appropriate range of bacterially expressed Odc1p and Odc2p standards (see Methods). In four determinations, the abundance of ODC proteins was 123±30 pmol/mg of protein of Odc1p, and 9±2 pmol/mg of protein of Odc2p.

The expression of Odc1p and Odc2p was investigated by immunoblot analysis of mitochondria isolated from the wild-type strain following growth on different carbon sources. The expression of Odc1p is repressed strongly by glucose whereas Odc2p appears to be expressed at comparatively higher levels on glucose and galactose media than on media supplemented with non-fermentable carbon sources (FIG. 6).

### Discussion

### Kinetic Properties of ODC Isoforms

The transport characteristics and kinetic parameters of the ODC proteins show that they are isoforms of a novel mitochondrial transporter for C5-C7 oxodicarboxylates with greatest specificity for 2-oxoadipate and 2-oxoglutarate. ODC also transports the corresponding dicarboxylates and to a lesser extent malate and citrate.

The substrate specificity of the yeast ODC isoforms is distinct from that of any other previously characterized mitochondrial carrier. It differs from that of the succinate-fumarate carrier (19), which is its closest sequence homologue (5), as the former transports fumarate and succinate with a very low efficiency (Km>15 mM). ODC is also quite different from the mammalian oxoglutarate carrier. First, the yeast ODC isoforms and the bovine oxoglutarate carrier have a sequence identity of 24 and 25%, indicating that they are not orthologues. Second, the ODCs transport C5-C7 oxodicarboxylates whereas the mammalian oxoglutarate carrier transports C4 and C5 oxodicarboxylates (14, 20, 21). Third, the ODC works best with C5-C7 dicarboxylates. whereas the mammalian oxoglutarate carrier displays optimal transport activity with C3 and C4 dicarboxylates (malonate, succinate and maleate) (14, 20, 21). Fourth, ODC appears to be less stereospecific than the mammalian oxoglutarate carrier as it has equal affinity for L- and D-malate, whereas the mammalian carrier has little or no affinity for the D-stereoisomer (14, 20, 21). Fifth, both isoforms of ODC accept the tricarboxylates, citrate and isocitrate as substrates, although with low affinity, whereas the specificity of the mammalian oxoglutarate carrier is confined to dicarboxylates (14, 20. 21).

### Regulation of Expression of the ODC Proteins

Many yeast genes involved in the tricarboxylic acid cycle, in oxidative phosphorylation and in ATP generation are subject to repression by fermentable carbon sources (catabolite repression). Under non-repressed conditions in cells fed on glycerol, Odc1p is about 15-fold more abundant than Odc2p. However, ODC1 expression is strongly repressed by catabolites, whereas ODC2 appears to be expressed at a higher level in the presence of galactose and glucose (FIG. 6). This apparent increase could result from the repression of most other mitochondrial proteins rather than from induction of the ODC2 itself. An increase in ODC1 expression has been observed without significant change in ODC2 expression during the diauxic shift when a culture of S. cerevisiae growing on glucose in batch culture exhausted the glucose supply and began to oxidize ethanol produced by fermentation (22). A temporal pattern of expression similar to that of ODC1 was observed for AAC1 and AAC2, the gene for the major “aerobic” isoform of ADP/ATP translocase, whereas the transcript level of AAC3, which is induced under anaerobic conditions, remained constant (22). These considerations indicate that Odc1p is the major ODC isoform under respiratory conditions and that Odc2p is the prevailing isoform in the presence of glucose and possibly in anaerobiosis. It should be noted that ODC1, AAC2 (encoding an isoform of ADP/ATP translocase) and MIR1 (encoding the phosphate carrier) are the only known yeast mitochondrial carrier genes that increase their expression following adaptive evolution in aerobic glucose-limited conditions (23), indicating that they all have key functions in cell metabolism.

### Role of ODC in Lysine Metabolism

In yeast, lysine is synthesized via the α-aminoadipate pathway, whereby 2-oxoadipate is produced in the mitochondrial matrix and 2-aminoadipate is converted into lysine in the cytoplasm (24). The results reported here suggest that 2-oxoadipate is exported by ODC from the mitochondrial matrix to the cytoplasm where it is transaminated to 2-aminoadipate (see FIG. 7). Since ODC functions by a strict exchange mechanism, the carrier-mediated efflux of 2-oxoadipate requires uptake of a counter-substrate. On the basis of transport measurements (Table II), 2-oxoglutarate, malate, or another transported Krebs cycle intermediate (according to the metabolic conditions) can fulfill this role and satisfy an important anaplerotic role by compensating the Krebs cycle for the 2)-oxoglutarate withdrawn for 2-oxoadipate synthesis.

Reversal of the cytoplasmic part of the 2-aminoadipate pathway is used in lysine catabolism in both S. cerevisiae and animals (24, 25). For this purpose, it is likely that 2-oxoadipate is imported by ODC into mitochondria first to be converted into glutaryl-CoA by 2-oxodipate dehydrogenase (a mitochondrial enzyme) and then metabolized in a series of steps to acetyl-CoA. In mammals, cytosolic 2-oxoadipate is also produced by catabolism of tryptophan (26) and possibly hydroxylysine. Therefore, it is likely that an ODC protein exists in man, and that defects in its activity could be linked to 2-ketoadipic acidemia. It has been suggested that this inborn error of catabolism of lysine, tryptophan and hydroxylysine (27) may be due to 2-oxoadipate dehydrogenase deficiency, but such a defect has not been demonstrated directly.

### Role of ODC in Nitrogen Assimilation

In S. cerevisiae, nitrogen in the form of ammonium is assimilated by the action either of isoforms 1 and 3 of glutamate dehydrogenase, or by glutamine synthetase and glutamate synthase together (28). These enzymes are cytoplasmic (29, 30) and nitrogen assimilation requires the carbon skeleton of 2-oxoglutarate. The major site of oxoglutarate production is mitochondrial matrix and therefore, another physiological role of ODC isoforms is probably to export it to cytoplasm. It should be stressed that it is unlikely that S. cerevisiae has an orthologue to the mammalian 2-oxoglutarate carrier (6). The only yeast carriers to cluster on a phylogenetic tree with the mammalian 2-oxoglutarate carrier (5) have been identified as the dicarboxylate and oxaloacetate carriers (4, 6). Since the odc1Δodc2Δ strain grew on different fermentable and non-fermentable carbon sources at rates similar to the parental strain, the mitochondrial ODC proteins are not indispensable for respiration, but this does not imply that the ODC is not involved in cytosolic glutamate formation. The synthetic media used in this study contained glutamate which may suffice to sustain the growth of mutant cells. Thus, more stringent growth conditions may be required to observe a phenotype. Also, yeast has alternative mechanisms for generating cytosolic 2-oxoglutarate to support nitrogen assimilation. It is possible that impairment of oxoglutarate export from mitochondria may be circumvented by the cytosolic NADP-dependent isocitrate dehydrogenase, Idp1p, which is sufficient for growth of S. cerevisiae without glutamate in the absence of the mitochondrial isozymes when oxoglutarate cannot be generated in the matrix (31). Another possibility is that yeast mitochondria contain a second unidentified oxoglutarate transporter.

### TABLE II—Dependence on Internal Substrate of the Transport Properties of Proteoliposomes Reconstituted with Recombinant Odc1p or Odc2p

Proteoliposomes were preloaded internally with various substrates (concentration, 20 mM). Transport was started by adding [¹⁴C]oxoglutarate (final concentration, 0.6 mM) and terminated after 45 seconds. Similar results were obtained in at least three independent experiments.

TABLE III
Effect of inhibitors on the [¹⁴C]oxoglutarate/oxoglfltarate
exchange by proteoliposomes reconstituted with Odc1p and Odc2p

[¹⁴C]oxoglutarate transport
(mmol/min per g protein)

| Internal substrate | Odc1p | Odc2p |
|---|---|---|
| None (CL present) | 0.4 | 0.2 |
| Oxaloacetate | 31.8 | 5.5 |
| 2-Oxoglutarate | 112.5 | 27.8 |
| 2-Oxoadipate | 85.0 | 22.1 |
| 2-Oxopimelate | 71.6 | 14.8 |
| Malonate | 4.8 | 2.2 |
| Succinate | 19.1 | 4.3 |
| Glutarate | 81.1 | 17.6 |
| Adipate | 65.0 | 16.7 |
| Pimelate | 48.4 | 10.4 |
| Suberate | 2.8 | 0.4 |
| L-malate | 56.4 | 11.4 |
| D-malate | 52.1 | 10.5 |
| Fumarate | 8.9 | 2.4 |
| Maleate | 7.1 | 2.6 |
| Citrate | 28.6 | 8.0 |
| L-Isocitrate | 19.4 | 3.7 |

Proteoliposomes were preloaded internally with 20 mM oxoglutarate and transport was initiated by the addition of 0.4 mM [¹⁴C]oxoglutarate. The incubation time was 45 seconds. Thiol reagents and α-cyanocinnamate were added 2 min before the labelled substrate; the other inhibitors and external substrates were added together with [¹⁴C]oxoglutarate. The final concentration of the inhibitors was 4 mM, except for mercurials (10 μM), carboxyatractyloside (0.1 mM), N-ethylmaleimide and α-cyanocinnamate (2 mM). Similar results were obtained in three independent experiments in duplicate.

TABLE IV
Competition with [¹⁴C]oxoglutarate uptake in proteoliposomes
containing recombinant yeast Odc1p and Odc2p.

Inhibition (%)

| Reagents | Odc1p | Odc2p |
|---|---|---|
| Mersalyl | 199 | 100 |
| p-Chloromercuriphenylsulfonate | 98 | 100 |
| HgCl₂ | 100 | 98 |
| N-Ethylmaleimide | 65 | 43 |
| Pyridoxal 5′-phosphate | 92 | |
| Bathophenanthroline | 97 | 89 |
| Butylmalonate | 118 | 25 |
| Phenylsuccinate | 23 | 22 |
| 1,2,3-Benzenetricarboxylate | 41 | 37 |
| α-Cyanocinnamate | 86 | 80 |
| Carboxyatractyloside | 7 | 10 |

The values were calculated from Lineweaver-Burk plots of the rate of [¹⁴C]oxoglutarate versus substrate concentrations. The competing substrates at appropriate constant concentrations were added together with 0.1-2.0 mM [¹⁴C]oxoglutarate to proteoliposomes containing 20 mM oxoglutarate and reconstituted with recombinant Odc1p or Odc2p. The data represent the means ±S.D. of at least three different experiments. n.d., not determined.

| | Ki (mM) | |
|---|---|---|
| Substrate | Odc1p | Odc2p |
| 2-oxoadipate | 0.26 ± 0.03 | 0.31 ± 0.04 |
| 2-oxopimelate | 1.83 ± 0.20 | 1.64 ± 0.18 |
| L-Malate | 1.32 ± 0.13 | 1.37 ± 0.16 |
| Citrate | 6.0 ± 0.8 | 5.9 ± 0.6 |
| Oxaloacetate | >15 | >15 |
| Succinate | >15 | n.d. |
| Isocitrate | >15 | n.d. |

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are apparent to those skilled in molecular biology or related fields are intended to be within the scope of the following claims.

### References

1. Palmieri, F., and van Ommen, B. (1999) in Frontiers Cellular Bioenergetics (Papa, S., Guerrieri, F., and Tager, J. M., eds.), Kluwer Academic/Plenum Publishers, New York, pp. 489-519

2. Walker, J. E. (1992) Curr. Opin. Struct. Biol. 2, 519-526

3. Palmieri, F. (1994) FEBS Lett. 346, 48-54

4. Palmieri, L., Palmieri, F., Runswick, M. J., and Walker, J. E. (1996) FEBS Lett. 399, 299-302

5. Palmieri, L., Runswick, M. J., Fiermonte, G., Walker, J. E., and Palmieri, F. (2000) J. Bioenerg. Biomembr. 32, 67-77

6. Palmieri, L., Vozza, A., Agrimi G., De Marco, V., Runswick, M. J., Palmieri. F., and Walker, J. E. (1999) J. Biol. Chem. 274, 22184-90

7. Palmieri, L., Lasorsa, F. M., Iacobazzi, V., Runswick, M. J., Palmieri, F., and Walker, J. E. (1999) FEBS Lett. 462, 472-476

8. Sherman, F. (1991) Methods Enzymol. 194, 3-21

9. Fiermonte, G., Palmieri, L., Dolce, V., Lasorsa, F. M., Palmieri, F., Runswick, M. J., and Walker, J. E. (1998) J. Biol. Chem. 273, 24754-24759

10. Daum, G., Gasser, S. M., and Schatz, G. (1982) J. Biol. Chem. 257, 13075-13080

11. Dulley, J. R., and Grieve, P. A. (1975) Anal. Biochem. 64,136-141

12. Laemmli, U. K. (1970) Nature 227, 680-685

13. Palmieri, F., Indiveri, C., Bisaccia, F., and Iacobazzi, V. (1995) Methods Enzymol. 260, 349-369

14. Palmieri, F., Quagliariello, E., and Klingenberg, M (1972) Eur. J. Biochem. 29, 408-416

15. Crompton, M., Palmieri, F., Capano, M., and Quagliariello, E. (1974) Biochem. J. 142, 127-137

16. Palmieri, F., Stipani, I., Quagliariello, E., and Klingenberg, M. (1972) Eur. J. Biochem. 26, 587-594

17. Klingenberg, M., Grebe, K., and Scherer, B. (1975) Eur. J. Biochem. 52, 351-363

18. Perkins, M., Haslam, J. M., and Linnane, A. W. (1973) Biochem. J. 134, 923-934

19. Palmieri, L., Lasorsa, F. M., De Palma, A., Palmieri, F., Runswick, M. J., and Walker J. E. (1997) FEBS Lett. 417, 114-118

20. Fiermonte, G., Walker, J. E., and Palmieri, F. (1993) Biochem. J. 294, 293-299

21. Bisaccia, F., Indiveri, C. and Palmieri, F. (1985) Biochim. Biophys. Acta 810, 362-369

22. DeRisi, J. L., Iyer, V. R., and Brown, P. O. (1997) Science 278, 680-686

23. Ferea, T. L., Botstein, D., Brown, P. O. and Rosenzweig, R. F. (1999) Proc Natl Acad Sci USA 96, 9721-9726

24. Bhattacharjee, J. K. (1985) Crit. Rev. Microbiol. 12, 131-151

25. Higashino, K., Fujioka, M., and Yamamura, Y. (1971) Arch. Biochem. Biophys. 142, 606-614

26. Nihizuka, Y., Ichiyama, A., and Hayaishi, O. (1970) Methods Enzymol. 17 part A, 463-487

27. Goodman, S. I., and Frerman, F. E. (1995) in The Metabolic and Molecular Basis of Inherited Disease (Scriver, C. R., Beaudet, A. L., Sly, W. S., and Valle, D., eds), McGraw-Hill, New York, Vol. I, pp. 1451-1460

28. Valenzuela, L., Ballario. P., Aranda, C., Filetici, P., and Gonzalez, A. (1998) J. Bacteriol. 180, 3533-3540

29. Hollemberg, C. P., Riks, W. F., and Borst, P. (1970) Biochim. Biophys. Acta 201, 13-19

30. Mitchell, A. P., and Magasanik, B. (1983) J. Biol. Chem. 258, 119-124

31. Zhao, W-N., and McAlisterHenn, L. (1996) Biochemistry 35, 7873-7878

1. A method for modulating transport of an oxodicarboxylate across the mitochondrial membrane of a yeast which method comprises modulating in said yeast the activity of one or more yeast mitochondrial transport polypeptides selected from:
(a). polypeptides comprising the amino acid sequence shown as SEQ ID NO:1 or homologues thereof; and
(b) polypeptides comprising the amino acid sequence shown as SEQ ID NO:2 or homologues thereof.

2. A method according to claim 1 which comprises expressing in said yeast, one or more nucleotide sequences encoding
(a) a polypeptide having the amino acid sequence shown as SEQ ID NO:1 or homologues thereof; and/or
(b) a polypeptide having the amino acid sequence shown as SEQ ID NO:2 or homologues thereof; and/or
(c) a fragment of the polypeptide of (a) or (b) having substantially the same activity.

3. A method according to claim 1 or 2 wherein said oxodicarboxylate is 2-oxoglutarate and/or 2-oxoadipate.

4. A method for increasing the rate of lysine biosynthesis in a yeast which method comprises modulating in said yeast the activity of one or more yeast mitochondrial transport polypeptides selected from:
(a) polypeptides comprising the amino acid sequence shown as SEQ ID NO:1 or homologues thereof; and
(b) polypeptides comprising the amino acid sequence shown as SEQ ID NO:2 or homologues thereof.

5. A method of producing a foodstuff which method comprises introducing into said foodstuff a yeast or product thereof which yeast has been modified by the method of claim 4.

6. Use of a yeast comprising a heterologous polypeptide which directs expression of one or more polypeptides selected from
(a) polypeptides having the amino acid sequence shown as SEQ ID NO:1 or homologues thereof;
(b) polypeptides having the amino acid sequence shown as SEQ ID NO:2 or homologues thereof; and
(c) fragments of the polypeptide of (a) or (b) having substantially the same activity; in the manufacture of a foodstuff.

7. A yeast produced by the method of any one of claims 1 to 4.

8. A foodstuff produced by the method of claim 5.


SEQUENCE LISTING (2 sequences)

SEQ ID NO: 1 - protein, 310 aa, Saccharomyces cerevisiae
MTSIDNRPLPFIYQFTAGAIAGVSELLVMYPLDVVKTRMQLQVTTKGHPAVVAAKAAVDHYTGVMDCLTKIVKKEGFSHLYKGITSPILMEAPKRAIKFSGNDTFQTFYKKIFPTPNGEMTQKIAIYSGASAGAVEAFVVAPFELVKIRLQDVNSQFKTPIEVVKNSVVKGGVLSLFNGLEATIWRHVLWNAGYFGIIFQIRKLLPAAKTSTEKTRNDLIAGAIGGTVGCLLNTPFDVVKSRIQRSSGPLRKYNWSLPSVLLVYREEGFKALYKGFAPKVMRLAPGGGLLLVVFTNVMDFFREVKYGKKQ

SEQ ID NO: 2 - protein, 292 aa, Saccharomyces cerevisiae
MSAEPLLPTHNGSQGGEVRSPDQKFIVIRFSDVSVRDLQLNISNVPFSNINTHWLRRMCRELRPQQTQKRRLKFIRNGSILNTHSKIAEELTHYFDTANNSNVATGTSVAPEQNNYYIHCIIGTEELTQAELANEDLKDDATPSNDSMTTQAIGFDRLRSVGFTEQEIELLRQQFRATYGDLEEEEERLAQNGNRDDEGHDIRQLEEQWMESGSGTAQGNGAGGGNEDRFNSVPIANIKHNKDLLLGICVGFFFGVFGILLMKFDGLFNRRQKMAIFAGVIVNVMFCLVRGF